(19) United States
(12) Patent Application Publication
(10) Pub. No.: US 2023/0070131 A1
(43) Pub. Date: Mar. 9, 2023

(54) GENERATING AND TESTING HYPOTHESES AND UPDATING A PREDICTIVE MODEL OF PANDEMIC INFECTIONS

(71) Applicant: Georgetown University, Washinton, DC (US)

(72) Inventors: Ophir Frieder, Chevy Chase, MD (US); David Hartley, Washington, DC (US)

(21) Appl. No.: 17/940,142

(22) Filed: Sep. 8, 2022

Related U.S. Application Data
(60) Provisional application No. 63/241,588, filed on Sep. 8, 2021.

(51) Int. Cl. G16H 50/80; G16H 50/20

(52) U.S. Cl. CPC G16H 50/80; G16H 50/20

(57) ABSTRACT

A system that generates and testing hypotheses about the spread of pandemic infections and updates a predictive model of the disease to reflect newly identified hypotheses and/or determinations that previously identified hypotheses are no longer suggested by the latest data. By coding, organizing, and sorting newly received data in a non-biased way, the disclosed system rapidly identifies new insights about the disease (and evidence challenging previously held assumptions about that disease) that can be communicated to public health officials, policymakers, and clinicians to better understand the nature of the disease and the effectiveness of clinical and public health interventions that are being used—or may be used—to control and treat the disease. The disclosed system also uses those new hypotheses (and evidence that previous hypotheses can be discounted) to adjust the predictive model to more accurately reflect the latest understanding of the disease and the effectiveness of potential interventions.

### CROSS-REFERENCE TO RELATED APPLICATIONS

This application claims priority to U.S. Prov. Pat. Appl. No. 63/241,588, filed Sep. 8, 2021, which is hereby incorporated by reference.

### FEDERAL FUNDING

None

### BACKGROUND

Pandemics of emerging infectious diseases happen aperiodically but not rarely. Examples include influenza pandemics in 1918 (influenza A/H1N1, “Spanish flu”), 1957 (influenza A/H2N2, “Asian flu”), 1968 (influenza A/H3N2, “Hong Kong flu”), 2009 (influenza 2009-H1N1/A), and coronaviruses in 2003 (SARS-CoV) and 2019 (SARS-CoV-2, the causative agent of COVID-19). Such events often carry significant morbidity and mortality globally.

Early in the spread of newly recognized or emergent pathogens, disease characteristics are often unknown and poorly understood in terms of transmission, agent durability in the environment, inoculation/infectious dose, host susceptibility, and—importantly— effective medical and public health control and intervention measures. Much in the same way as the recognized phases of other natural disasters, a hallmark of newly emergent diseases is that early information is often confused, limited, incorrect, and skewed. In the case of SARS-CoV-2, for example, early information on COVID-19 implicated highest risk for older adults with comorbid conditions, producing the assumption/implication that younger persons were not at risk for severe disease and death. That assumption has proven tragically untrue.

Other assumptions that have proven untrue over time in the COVID-19 experience include that the disease is mild in adults under the age of 65, that COVID-19 is transmitted by droplets and not aerosols, the efficacy of masks in preventing transmission, and that vaccinated persons do not shed meaningful concentrations of virus and, therefore, do not participate in the disease transmission cycle.¹ Reliance on those incorrect assumptions has proven to be a significant impediment to effective control and management of the COVID-19 pandemic in the United States and elsewhere. ¹ Barker, Hartley, Beck et al, Rethinking Herd Immunity Managing the Covid-19 Pandemic in a Dynamic Biological and Behavioral Environment, NEJM Catalyst, 10 Sep. 2021, https://catalyst.nejm.org/doi/full/10.1056/CAT.21.0288

For newly emerged infections, what is learned early from a small number of observations often influences decisions in other circumstances incorrectly. In the case of COVID-19, for example, the Wuhan experience suggested controls that apparently worked in China but were later shown to be inaccurate.² Nevertheless, the mistaken belief that those controls were effective influenced U.S. policies and thinking regarding interventions.³ ² See, e.g., Pan et al, Association of Public Health Interventions with the Epidemiology of the COVID-19 Outbreak in Wuhan, China, JAMA, 19 May 2020, https://pubmed.ncbi.nlm.nih.gov/32275295/; Hartley and Perencevich, Public Health Interventions for COVID-19: Emerging Evidence and Implications for an Evolving Public Health Crisis, JAMA, 19 May 2020, https://pubmed.ncbi.nlm nih gov/32275299/³ Auger, Shah, Richardson, Hartley et al, Association Between Statewide School Closure and COVID-19 Incidence and Mortality in the US, JAMA, 1 Sep. 2020, https://pubmed.ncbi.nlm.nih.gov/32745200/

Accordingly, to identify effective medical and public health control and intervention measures in the emergent stages of each pandemic, it is vitally important to correctly ascertain the characteristics of a novel disease and the effectiveness of each measure.

Additionally, the coronavirus pandemic revealed the extent to which policymakers rely on predictive models, which attempt to predict the future of virus spread, to decide what actions are best to take.⁴ Although better than relying on intuition or flying completely blind into a crisis, predictive models rely on assumptions about disease characteristics and the effectiveness of public health and medical interventions. For instance, of the 28 probabilistic forecasts evaluated in a recent paper, seven made explicit assumptions that social distancing and other behavioral patterns would change over the prediction period.⁵ As additional information is collected over time, the data may challenge or contradict some of the assumptions used by those predictive models to predict future virus spread. Additionally, emerging data may suggest additional elements that, if incorporated into the predictive model, would improve the accuracy of the predictive model. Reliance on COVID-19 models that failed to adjust in view of new ⁴ Sample, I., Coronavirus exposes the problems and pitfalls of modelling, The Guardian 2020 Mar. 25, https://www.theguardian.com/science/2020/mar/25/coronavirus-exposes-the-problems-and-pitfalls-of-modelling⁵ Cramer et al, Evaluation of individual and ensemble probabilistic forecasts of COVID-19 mortality in the United States, PNAS, 8 Apr. 2022, https://doi.org/10.1073/pnas.2113561119 evidence may have led to several missteps.⁶ For example, some early COVID-19 models did not consider the possible effects of mass “test, trace, and isolate” strategies or potential staff shortages on transmission dynamics.⁷ Including those factors in predictive models may have led to an earlier focus on testing capacity and providing appropriate protective equipment for frontline workers. Accordingly, correctly ascertaining the characteristics of a novel disease and the effectiveness of interventions is also vitally important when modeling the future spread of the novel disease.⁶ Ahmed, N., Covid-19 special investigation, part 1, The politicized science that nudged the Johnson government to safeguard the economy over British lives, Byline Times 2020 Mar. 23, https://bylinetimes.com/2020/03/23/covid-19-special-investigation-part-one-the-politicised-science-that-nudged-the-johnson-government-to-safeguard-the-economy-over-british-lives/⁷ Sridhar et al., Modelling the pandemic, BMJ 21 Apr. 2020, https://doi.org/10.1136/bmj.m1567

Newly emergent “learning health systems” and “learning networks,” which rapidly learn from data and disseminate learning to system stakeholders, are regarded as an important advance in US healthcare.⁸ That advance has the potential to rapidly disseminate critical information in emergent pandemic situations, but also runs the risk of promulgating incorrect conclusions and information. Currently lacking in the art but critically needed—especially in the case of pandemics⁹—is the ability to learn rapidly in a non-biased way, revise that learning as new data are observed, and rapidly communicate insights to stakeholders such as hospitals and public health departments throughout medicine. More so, that ability must support the identification of new insights that challenge or contradict previous conclusions and assumptions as additional information is obtained. ⁸ Ardura, Hartley, Dandoy et al, Addressing the Impact of the Coronavirus Disease 2019 (COVID-19) Pandemic on Hematopoietic Cell Transplantation: Learning Networks as a Means for Sharing Best Practices, Biol Blood Marrow Transplant, July 2020, https://pubmed.ncbi.nlm.nih.gov/32339662/⁹ Beck, Hartley, Kahn et al, Rapid, Bottom-Up Design of a Regional Learning Health System in Response to COVID-19, Mayo Clin Proc, 16 Feb. 2021, https://pubmed.ncbi.nlm nih gov/33714596/; Hartley, Beck, Seid et al, 16. Multi-sector Situational Awareness in the COVID-19 Pandemic: The Southwest Ohio Experience, 2021, https://www.springerprofessional.de/en/multi-sector-situational-awareness-in-the-covid-19-pandemic-the-/19551082

An especially important need exists in the area of pandemic detection and early warning,¹⁰ currently a major focus of interest.¹¹ That can be seen in the case of Project Argus,¹² which examined massive amounts of unstructured, multilingual textual data to detect leading indicators of infectious disease outbreaks globally. Project Argus made observations of disease or potential disease incidents, enabling human analysts to form hypotheses regarding the correct interpretation of such events on an ad hoc basis. Importantly, those hypotheses often changed over the course of days to weeks to months as events evolved and spread, and as additional data became available. More recently, systematic machine methods were developed that generate and rank a universe of relevant hypotheses.¹³ However, there was no systematic way to test the assumptions made earlier in the assessment of a novel disease and determine whether, as data emerge over time, the newly received data challenge or contradict those assumptions. ¹⁰ Nelson, Brownstein, and Hartley, Event-based biosurveillance of respiratory disease in Mexico, 2007-2009: connection to the 2009 influenza A(H1N1) pandemic?, Euro Surveill, 29 Jul. 2010, https://pubmed.ncbi.nlm nih gov/20684815/; Hartley, Nelson, Arthur et al, An overview of internet biosurveillance, Clin Microbiol Infect, 19 Nov. 2012, https://pubmed.ncbi.nlm.nih.gov/23789639/¹¹ CDC Stands Up New Disease Forecasting Center, https://www.cdc.gov/media/releases/2021/p0818-disease-forecasting-center.html¹² Hartley et al, Landscape of international event-based biosurveillance, Emerg Health Threats, 19 Feb. 2010, https://pubmed.ncbi.nlm nih gov/22460393/; U.S. Pat. No. 10,002,034 to Li, Torii, Hartley and Nelson¹³ e.g., U.S. Pat. Nos. 10,521,727 and 11,106,878 to Frieder and Hartley; Parker, Wei, Yates, Frieder and Goharian, A framework for detecting public health trends with Twitter, Proceedings of the 2013 IEEE/ACM International Conference on Advances in Social Networks Analysis and Mining, 25 Aug. 2013, https://dtacm.org/doi/10.1145/2492517.2492544

The uncertainty associated with early assumptions regarding pandemics is often not recognized. That uncertainty may stem from imprecise reporting, unintentional (and, at times, intentional) misleading information, political agendas, general lack of understanding, incomplete information due to the novelty of the pathogen, etc. Thus, a need exists to combine uncertain information based on early observations with new observations as disease spreads to new areas to avoid being misled and surprised.

### SUMMARY

The disclosed system addresses the problem of how to transform what is learned from early observations into new information based on data in new areas to which emerging infections spread, resulting in forecasts and interventions tailored to local areas (e.g., villages, towns, cities, counties, zip codes, states, countries, etc.) as well as updated guidelines. The disclosed system learns more rapidly than is possible at present by iteratively combining data from multiple sources (using machine learning based on massive longitudinal and geographic data collection and data fusion). The disclosed system securely manages and resolves data conflict (e.g., noise effect reduction) to support an epidemic response tailored to local circumstances and populations (i.e., precision public health). The disclosed system supports the extraction, integration, and reconciliation of multiple local population segments to yield, analyze, propagate, and disseminate global guidelines.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a block diagram of an architecture of a system for generating and testing hypotheses and updating a predictive model of pandemic infections according to an exemplary embodiment.

FIG. 2 is a block diagram illustrating the system generating initial hypotheses and an initial prediction using initial data according to an exemplary embodiment.

FIG. 3A is a flowchart illustrating a process for generating the initial hypotheses using the initial data according to an exemplary embodiment.

FIG. 3B is a flowchart illustrating the process for generating updated hypotheses using the updated data according to an exemplary embodiment.

FIG. 4 is a block diagram illustrating the system generating and distributing the updated hypotheses and an updated prediction according to an exemplary embodiment.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

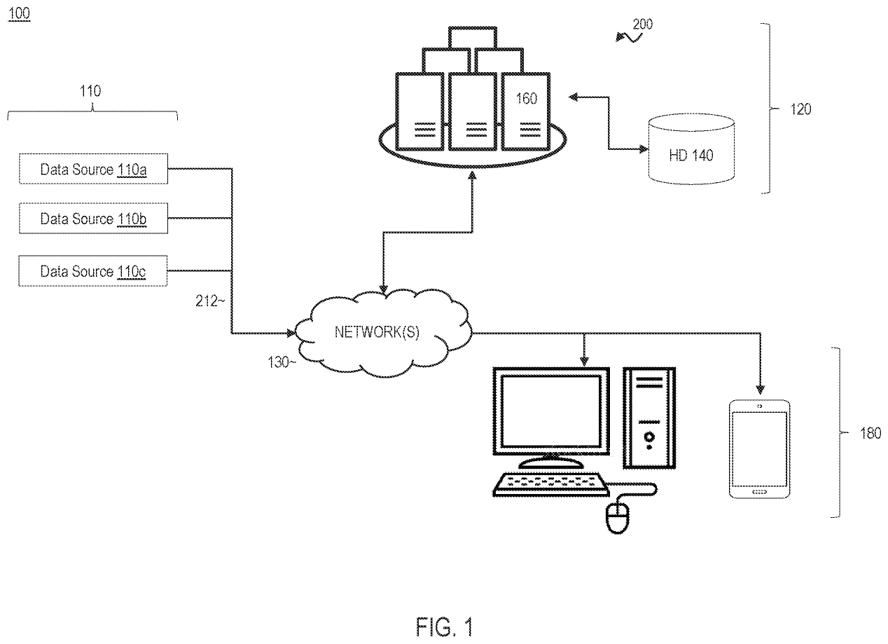

FIG. 1 is a block diagram of an architecture 100 of a system 200 for generating and testing hypotheses and updating a predictive model of pandemic infections according to an exemplary embodiment.

As shown in FIG. 1, the architecture 100 may include a server 120 that communicates with client devices 180, for example via one or more networks 130 such as the Internet. The server 120 includes one or more hardware computer processors 160 and non-transitory computer readable storage media 140. The server 120 receives data 212 from data sources 110. The server 120 may be any suitable computing device including, for example, an application server or a web server. As described below, in some embodiments the data 212 may be publicly available and the data sources 110 may be accessible via the Internet. In other embodiments, some of the data 212 may be proprietary and/or sensitive. In those embodiments, the server 120 may be the secure computing environment for co-analyzing proprietary data, for example described in U.S. patent application Ser. No. 16/663,547, which is hereby incorporated by reference.

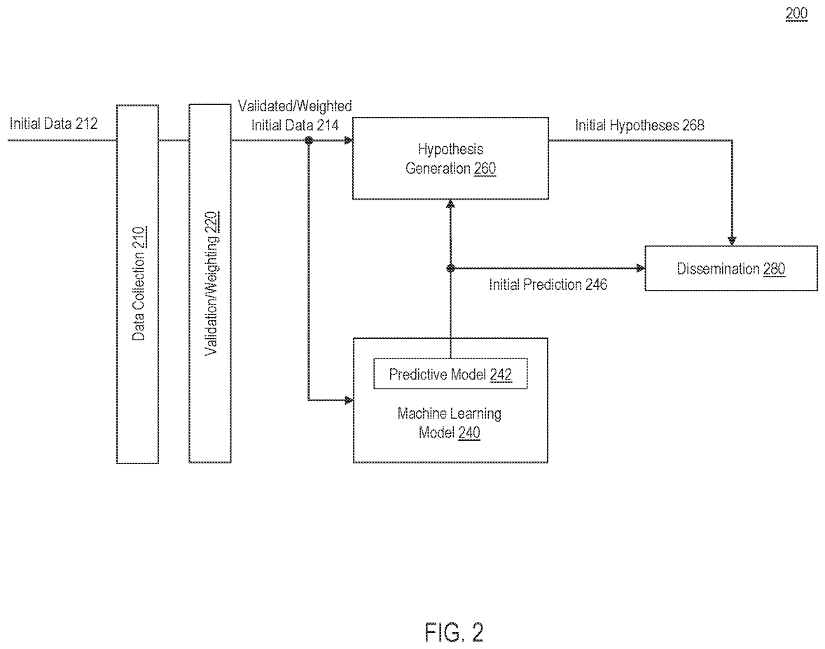

FIG. 2 is a block diagram illustrating the system 200, which is realized by software modules executed by the hardware computer processor(s) 160, generating and distributing initial hypotheses 268 and an initial prediction 246 according to an exemplary embodiment.

As shown in FIG. 2, the system 200 may include a data collection module 210, a validation/weighting module 220, a machine learning module 240, a hypothesis generation module 260, and a dissemination module 280.

The data collection module 210 collects the data 212 from the data sources 110. The data collection module 210 may perform web crawling, scraping, and/or proprietary data ingestion and may include structured data connectors, for example as described in U.S. Pat. No. 10,002,034, which is hereby incorporated by reference. In some embodiments, data ingestion may rely on extract, transform, and load (ETL) or other data cleansing techniques known in the art. In some embodiments, the data collection module 210 may be configured to collect data 212 in multiple languages and translate that data 212. In some other embodiments, the data collection module 210 may be configured to collect multimedia data 212 and extract features and textualize those data 212.

The data 212 may include information in a variety of formats from a variety of data sources 110. For instance, the data 212 may include spatio-temporal data regarding a disease, symptoms of that disease, human behavior contemporaneous with and/or in response to the disease, environmental conditions, meteorologic conditions, demographic and/or cultural data regarding geographical areas, and other data types in textual, numeric, image, and other formats. The data 212 may include structured or unstructured alphanumeric and non-alphanumeric elements, grammatically or ungrammatically structured text, non-text components (e.g., tables, figures, annotations, logos, images, or other element conveying information). The data 212 may include composite data (e.g., graphs, charts, spreadsheets, etc.). The data 212 may include medical and scientific literature (e.g., published peer reviewed studies including rapid review), open-source information (e.g., social media posts, public health reports, etc.), etc. The data 212 may include electronic health records from health information exchanges, regional health information organizations (e.g., The Health Collaborative,¹⁴ Chesapeake Regional Information System for our Patients,¹⁵ etc.), etc. The data 212 may include an aggregation of data collected from wearable devices (activity or fitness trackers, smart watches, etc.), personal communication devices (e.g., smartphones), Internet of Medical Things (IoMT) devices (e.g., remote patient monitoring devices, medication trackers, etc.), etc. ¹⁴ https://healthcollab.org/¹⁵ https://crisphealth.org/data

The validation/weighting module 220 validates the data 212 and assigns a weight to each document in the data 212 to form and output validated and weighted data 214. While all of the data 214 may be of interest, some of the data 214 may have different associated weights depending on characteristics of the data 214 such as the nature, source of capture, volume, uniqueness, and variance of the data 214. Additionally, documents in the data 214 may be weighted based on the quality of the source of that data 214 (e.g., trustworthiness, authority, target audience, writing/reading level, number of references cited, domain of interest, etc.). As such, some documents in the data 214 may be treated as being more valuable than others. For instance, the validation/weighting module 220 may assign a higher weight to a study published in the Lancet or a Weekly Epidemiological Report from the World Health Organization than a blog post.

In some embodiments, the validation/weighting module 220 may weight data 214 from data sources using existing (qualitative or quantitative) measures of the reliability of those sources, such as the impact factor of a journal (a measure of the frequency with which the average article in the journal has been cited in a particular year), a reputation score of a website as determined by a web reputation service,¹⁶ etc. ¹⁶ e.g., https://www.brightcloud.com/tools/url-ip-lookup.php

In emergent situation like a pandemic, however, data sources may emerge that are not rated by existing measures of reliability but nevertheless provide valid, reliable data 214.¹⁷ Accordingly, in some embodiments, the validation/weighting module 220 may weight data 214 using heuristics and/or subjective determinations of the reliability of specific data sources. For example, the validation/weighting module 220 may store a table of trusted data sources and weight the data 214 from those trusted data sources higher than data 214 received from other data sources. For instance, the validation/weighting module 220 may weight the data 214 from each of those trusted data sources equally or may store individual weights for each trusted data source (that are all higher than weights applied to data 214 received from other data sources). Similarly, the validation/weighting module 220 may store a table of data sources considered untrustworthy and either de-weight the data 214 from those untrustworthy data sources or may invalidate and ignore the data 214 from those untrustworthy data sources. In those embodiments, the validation/weighting module 220 may provide functionality for authenticated users (i.e., subject matter experts) to specify trustworthy and untrustworthy data sources (e.g., journals, epidemiological data sources, etc.) identified using their preferred criteria (e.g., transparency, reliability of past data, or other criteria). By providing functionality to identify trustworthy data sources (and, in some instances, weights to apply to data 214 received from those trustworthy data sources), the validation/weighting module 220 enables subject matter experts to weight data 214 using new criteria that may suggest itself in the moment. For instance, data 214 from health exchange organizations may be rated very highly because those health exchange organizations have access to electronic health records. ¹⁷ e.g., the Covid Tracking Project (https://covidtracking.com/about), the COVID-19 School Data Hub (https://www.covidschooldatahub.com), etc.

### Modeling Pandemic Infections

Using the initial data 214 provided by the validation/weighting module 220, the machine learning module 240 develops a predictive model 242 that predicts the future spread of the disease based on the data 214 output by the validation/weighting module 220 and associations, identified by the machine learning module 240, between predictor variables that are identifiable in the data 214 and a dependent variable. For example, the predictive model 242 may predict the magnitude of one or more disease-related metrics (e.g., infections, hospitalizations, deaths, etc.) by applying weights and biases to predictor variables included in the data 214. In another example, the predictive model 242 may be a probabilistic model (e.g., a Bayesian belief network) that calculates the probability of one or more disease events based on associations between predictor variables included in the data 214 and the probability of those future disease events.

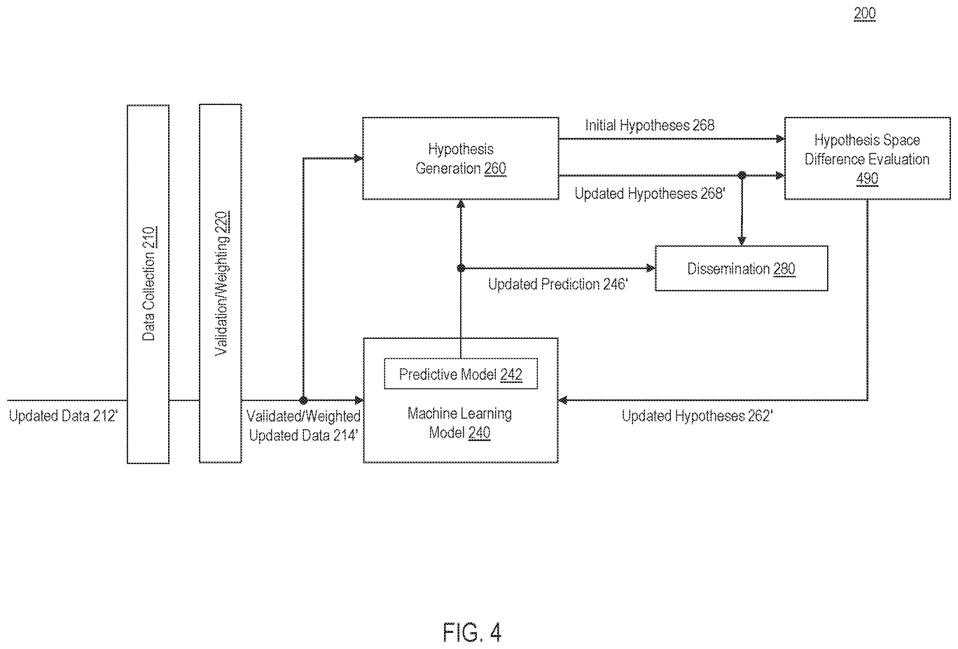

The predictive model 242 generated by the machine learning module 240 may be a machine learning model, a mathematical model (e.g., an epidemic model, a contagion model, a hospital needs model, etc.), etc. As shown in FIGS. 2 and 4, the predictive model 242 uses the data 214 output by the validation/weighting module 220 to generate predictions 246 regarding the spread of the disease. The predictions 246 may be probabilistic or deterministic forecasts of the magnitude of a dependent variable (e.g., an infection rate, hospital capacity, availability of personal protective equipment, etc.), the probability of a dependent variable (e.g., a disease-related event), etc. The predictor variables identified by the machine learning module 240 as associated with the dependent variable may include numerical values (having magnitudes, rates over time, rates of change, rates of acceleration, ratios relative to other numeric variables, etc.), whether certain conditions are true or false (e.g., whether certain public health interventions have been implemented, etc.), etc. The associations, identified by the machine learning module 240, between the predictor variables and the dependent variable may include any predictive associational relationships and/or causal relationships between the predictor variables and the dependent variable, including correlations between the predictor variables and the dependent variable, non-linear mappings of the predictor variables onto the dependent variable, and/or any other relationships between the predictor variables onto the dependent variable.

To generate the predictive model 242, the machine learning module 240 is trained using the data 214 to learn both the predictor variables in the data 214 that may be associated with the future spread of the disease and the associations (e.g., weights, Bayesian probabilities, etc.) between those predictor variables and the predicted disease-related metric or event. The machine learning module 240 may utilize any or all supervised, unsupervised, or semi-supervised learning approaches. The machine learning module 240 may utilize approaches that include classification, regression, regularization, decision-tree, Bayesian, clustering, association, neural networks, deep learning algorithms, etc. Deep learning algorithms may include recurrent models, convolutional models, transformer models with or without attention, etc. The machine learning module 240 may employ various machine learning algorithms known in the art, for instance pre-train transformers (used as global data), one or more final layers (trained while maintaining previous layers for localization),¹⁸ etc. ¹⁸ MacAvaney, Nardini, Perego, Tonellotto, Goharian, and Frieder, Efficient Document Re-Ranking for Transformers by Precomputing Term Representations, ACM Forty-Third Conference on Research and Development in Information Retrieval (SIGIR), July 2020, https://dl.acm.org/doi/abs/10.1145/3397271.3401093

As shown in FIG. 2, the predictive model 242 uses the initial data 214 to generate an initial prediction 246 of how the disease will spread in one or more locations.

### Generating and Testing Hypotheses

Using the initial data 214 provided by the validation/weighting module 220, the hypothesis generation module 260 generates a ranked list of initial hypotheses 268.

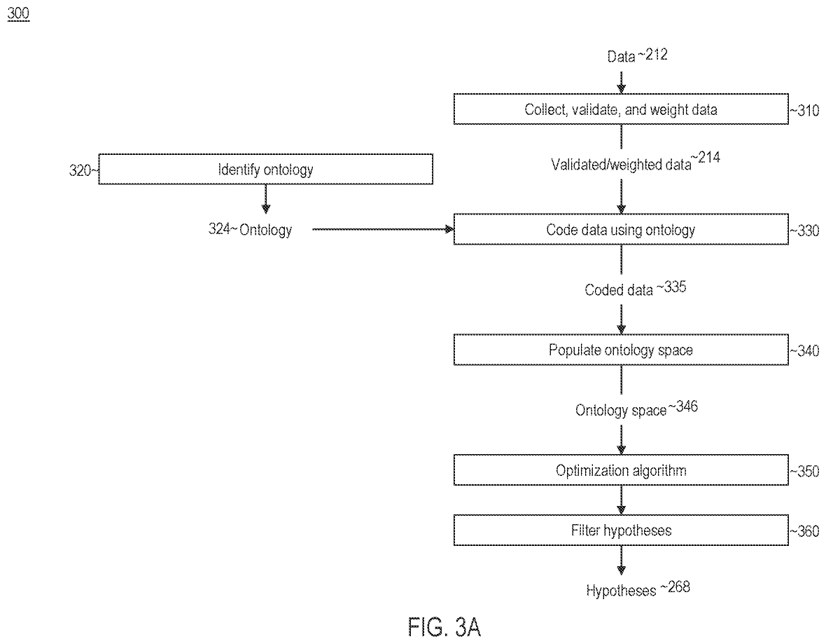

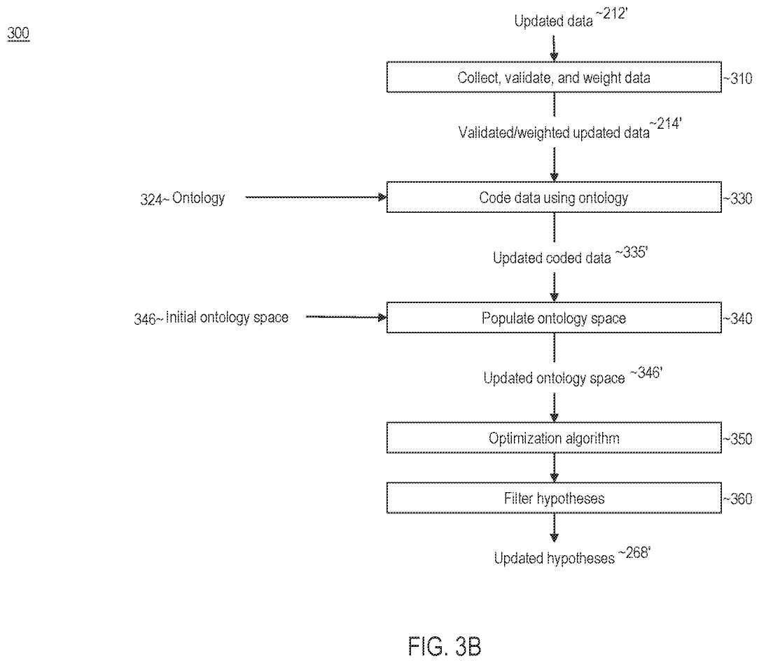

FIGS. 3A and 3B are flowcharts of a hypothesis generation process 300 according to an exemplary embodiment.

As shown in FIG. 3A, the initial data 212 are collected from the data sources 110 in step 310 as described above. In some embodiments, each document in the data 212 is validated and weighted to form validated and weighted data 214 as shown in FIG. 3A.

An ontology 324 is identified in step 320. An ontology 324 is a set of possible event descriptions. That ontology can be understood to represent a formal conceptualization of a particular domain of interests or a definition of an abstract view of a world a user desires to present. Such conceptualization or abstraction is used to provide a complete or comprehensive description of events, interests, or preferences from the perspective of a user who tries to understand and analyze a body of information.

Each ontology 324 includes a number of elements. An ontology 324 with three elements, such as {subject, verb, object} for example, is used to detect all data corresponding to the notion “who did what to whom.” A 6-element ontology 324 may include {what, who, where, indicators, actions, consequences}. Each element includes choices of terms for that element of the ontology 324, known as a “vocabulary.” If each element in a 6-element ontology 324 has a 100-term vocabulary, for example, then the ontology 324 defines 100⁶ descriptions of distinct, mutually exclusive (although possibly related) events. Accordingly, the ontology 324 constitutes the set of all distinct combinations of hypotheses considered during the hypothesis generation process 300. Each combination of elements in an ontology 324 is referred to as a “ontological vector.”

For many vocabulary terms, synonyms exist that refer to the same real-world concept. Accordingly, the ontology 324 may include synonym collections that each correspond to one of the vocabulary terms.

The ontology 324 may be supplied by a user or may be constructed by the system 200 using datasets being analyzed using machine methods. The ontology 324 identified in step 320 is preferably specific to an infectious disease. Accordingly, a subject matter expert (SME) preferably vets the ontology 324 to ensure that it accurately represents the domain knowledge of the data 214 under consideration.

The data 214 are coded using the ontology 324 to form coded data 335 at step 330. Specifically, the computer processor(s) 160 executing the hypothesis generation module 260 search the data 214 using one or more entity extraction schemes that are known in the art to determine which ontological vectors in the ontology 324 appear in the data 214. Each ontological vector identified in the data 214 represents a hypothesis 268. For example, an analysis of reports on public health using a 3-element {subject, verb, object} ontology 324 may identify the following ontological vectors representing the following hypotheses:

1. Virus causes pneumonia.

2. Bacterial illness causes death.

3. Influenza-like illness causes unknown.

In some embodiments, the hypothesis generation module 260 also assigns each ontological vector identified in the data 214 to the corresponding elements of text in the data 214 that include the ontological vector.

The ontology 324 can be graphically represented as an ontology space 346, for example with as many dimensions as there are elements in the ontology 324. The ontological vectors identified in the data 214 form an ontology space 346 at step 340. A one-element ontology 324, for example, forms an ontology space 346 with only one dimension (i.e., a line), which is readily understandable by a human analyst. Each point along the line represents a vocabulary term in the ontology 324. It can be imagined that each time a vocabulary term is identified in the data 214, a bar graph at that point along the line gets higher (or lower). The vocabulary terms found most often in the data 214 are represented by the highest peaks (or lowest troughs) along the one-dimensional ontology space 346. Two-element and three-element ontologies 324 may form two-dimensional and three-dimensional ontology spaces 346, which are more complicated but may still be visualized and comprehended by an analyst. However, when the ontology 324 has more than three elements and forms a 4-dimensional, 5-dimensional, or even 100-dimensional ontology space 346, the ontology space 346 becomes so complex that no human analyst could ever intuitively understand it.

Regions of the initial ontology space 346 are populated as the documents in the data 214 are coded. The populated ontology space 346 is a geometric representation of possible events that are encoded by that particular corpus of data 214 according to that particular ontology 324. The ontological vectors identified in the data 214, which are assigned to the corresponding coordinates in the ontology space 346, form structures in the ontology space 346. In particular, points in the ontology space 346 that are populated by successive occurrences in the data 214 are assigned a value corresponding to a larger weight (described above as a higher peak or lower trough) than points in the ontology space 346 that are found less often in the data 214. When all documents are coded, the ontology space 346 is populated by clusters (i.e., neighborhoods of points) of differing weights. The clusters of points of highest weight in the ontology space 346 correspond to the most likely hypotheses of what the data 214 are describing.

As described above, an ontology 324 with N elements may be depicted graphically in an N-dimensional ontology space 346, where each dimension of the N-dimensional ontology space 346 represents one of the N elements of the ontology 324. In other embodiments, however, the hypothesis generation module 260 may perform dimension reduction such that the ontology space 346 has fewer dimensions than the number of elements in the ontology. For example, the hypothesis generation module 260 can separate the N elements of the ontology 324 into R groups and then depict them graphically in the coded data 335 in an R-dimensional ontology space 346. Depending on the nature of the ontology 324, the hypothesis generation module 260 may perform lossless dimension reduction to preserve semantic content or perform dimension reduction with an acceptable loss across dimensions.

As described above, the data 214 may be weighted by the validation/weighting module 220 based on characteristics of the data 214 (e.g., the source, nature, volume, uniqueness, and variance of the data 214). Accordingly, the hypothesis generation module 260 may weight each of the ontological vectors identified in the data 214 based on the weight of the data 214 from which each ontological vector was identified. Additionally, each attribute of the ontology 324 may be weighted based on the significance of that attribute. For example, attention may be placed on one or more dimensions of the ontology space 346 to place additional weight on the magnitude of each ontological vector along those one or more dimensions. Additionally, within each attribute of the ontology 324, some of all of the vocabulary terms may be weighted based on the significance of those vocabulary terms. For example, the hypothesis generation module 260 may assign higher weights to ontological vectors that include more specific vocabulary terms than ontological vectors that include more generic vocabulary terms. Additionally, as described in U.S. Pat. No. 11,106,878, ontological vectors may be weighted based on the profile of a particular user. For example, if a user is interested in Asia and not Africa, ontological vectors with Africa as a component may be de-valued or excluded. Alternately, ontological vectors with Africa as a component may could be weighted more heavily as they may suggest connections to foreign nations that are of interest.

The hypothesis generation module 260 may also group or merge ontological vectors describing similar or related concepts into neighborhoods in the ontology space 346. For example, the hypothesis generation module 260 may identify ontological vectors that describe similar or related concepts—for example, {masks, prevent, new infections} and {masks, stop, viral spread}— that are not distinct events. If the ontology 324 is ordered, meaning similar or related choices for each ontology element appear in order, the similar or related ontological vectors in the coded data 335 will appear close together in the ontology space 346. That is, the embeddings or representations of the coded data 335 will map to a near vicinity, i.e., neighborhood, within the ontology space 346. In one embodiment, the embeddings or representations of the coded data 335 will map to a near vicinity, i.e., neighborhood, within the ontology space 346. Accordingly, the hypothesis generation module 260 may merge similar and/or related ontological vectors (e.g., via clustering hierarchies, filters/thresholds, topic models, conditional random fields, deep learners, etc.).

An optimization algorithm identifies hypotheses 268 in the ontology space 346 populated by the ontological vectors found in the data 214 (and ranks those identified hypotheses 268) at step 350. The computer processor(s) 160 executing the hypothesis generation module 260 identify and rank the hypotheses 268 by identifying the clusters of highest weights in the ontology space 346. Identifying that set of clusters in the ontology space 346 is not a trivial problem for ontologies 324 of significant size and structure. However, it is a moderately well-defined optimization problem that can be solved using an iterative optimization algorithm (such as coordinate or gradient descent) or a heuristic optimization algorithm (such as simulated annealing, a Monte Carlo-based algorithm, a genetic algorithm, etc.).

Simulated annealing, for example, identifies the highest weighted clusters in an efficient and robust manner by selecting a random point in the ontology space 346 and letting simulated annealing govern a random “walk” through the weighted ontology space 346 via a large number of heat-cooling cycles. The computer processor(s) 160 executing the hypothesis generation module 260 build up an ensemble of such cycles for a large number of randomly chosen initial points. An accounting of the most highly weighted regions in the weighted ontology space 346 then corresponds to a ranked list of the hypotheses 268 that potentially explain the material in the data 214, which may be presented to an analyst to test. In another example, the ontology space 346 can graphically depict populations and a genetic algorithm can be used to identify and rank the highest weighted ontological vectors or neighborhoods in terms of fitness of population.

In some instances, the dataset of ontological vectors identified in the data 214 may be so numerous that it is impractical or even infeasible for the server 120 to rank each ontological vector (or group of similar or related ontological vectors) using a computationally intensive optimization routine. Accordingly, in some embodiments the hypothesis generation module 260 may use a first optimization function to perform a coarse ranking of the ontological vectors or groups and a second optimization function to perform a more precise ranking of the ontological vectors or groups ranked highest by the first optimization function. In some of those embodiments, the hypothesis generation module 260 may use a first optimization function (e.g., a heuristic optimization function) that is less computationally intensive than the second optimization function to process the entire dataset of ontological vectors or groups and a second optimization function (e.g., an iterative optimization function) that is more computationally intensive than the first optimization algorithm to process the smaller subset of ontological vectors or groups ranked highest by the first optimization function. In those instances, using a first optimization function that is less computationally intensive routine to perform the coarse ranking may make the process of ranking the entire dataset of hypotheses 268 tractable for the server 120. Meanwhile, reducing the amount of data needed to be examined in detail may make it tractable for the server 120 to use a second, more computationally intensive optimization routine to refine and improve the accuracy of the coarse ranking. In other embodiments, both optimization functions may be of similar complexity but functionally differ. Therefore, using an optimization algorithm that includes two separate optimizations functions may enable the hypothesis generation module 260 to both process the entire dataset of ontological vectors identified in the data 214 while also accurately and precisely ranking the hypotheses 268 in accordance with the weight of their associated ontological vectors (or groups of similar or related ontological vectors).

In some embodiments, the hypothesis generation module 260 may rank the hypotheses 268 based on the weight of each ontological vector or group of similar or related ontological vectors (e.g., using the first optimization function as described above), adjust the weight the ontological vectors or groups (e.g., by placing attention on one or more dimensions of the ontology space 346 to place additional weight on the magnitude of each ontological vector along those one or more dimensions as described above), and re-rank the hypotheses 268 according to the adjusted weights of the ontological vectors or groups corresponding to those embodiments (e.g., using the second optimization function as described above).

The hypotheses 268 may be filtered at step 360 to generate a filtered set of ranked relevant hypotheses 268. Trivial hypotheses (such as tautologies) and/or nonsensical hypotheses may be discarded. Techniques from information retrieval and natural language procession (e.g., term frequency, scope and synonym analysis, etc.) may be used to identify and discard trial and/or nonsensical hypotheses. A hypothesis 268 that only contains frequent words, for example, is most likely too general to be of interest. In some embodiments, additional weighting can be placed on particular dimensions to rescore and possibly reorder the hypotheses 268.

Local minima effects can sometimes provide a solution even when a better solution exists in another neighborhood. Random variations or mutations in the optimization algorithm (e.g., simulated annealing or genetic process) can be used to prevent the incorrect determination of a desired solution (e.g., a hypothesis of limited value) due to local minima effects. Those variations or mutations may be guided. At each proposed mutation, the neighborhood can be assessed for fitness. In an annealing process, for example, fitness can be assessed by the rate of change (e.g., the slope of descent or accent). In a genetic process, the fitness of a population member can be computed. In either process, a mutation can be rejected if the mutation results in an ontology space 346 that is deemed highly anticipated. Additionally, the rate of mutation can be modified to be a function of the anticipation level of the neighborhood initially in (e.g., a nonlinear mapping, a simple proportional dependence, etc.). Still further, the level of anticipation can be based on the profile of the analyst receiving the hypotheses.

The hypothesis generation module 260 may determine and output a degree of certainty as to the likelihood of each generated hypothesis 268. The degree of certainty as to the likelihood of each generated hypothesis 268 is related to the confidence in—and support for—each generated hypothesis 268. The hypothesis generation module 260 may determine a degree of certainty for each hypothesis 268 based on (e.g., proportional to) the weight ontological vector or neighborhood associated with that hypothesis 268, which is based on (e.g., proportional to) the number of documents within the data 214 (and the weight of those documents) that, when coded, are found to contain the ontological vector or an ontological vector within that neighborhood.

As alluded to above, the system 200 repeatedly performs the hypothesis generation process 300 to generate updated hypotheses 268′ based on updated data 214′ (and discard initial hypotheses 268 that are no longer supported by the updated data 214′). As shown in FIG. 3B, updated data 212′ are collected (and, in some embodiments, validated and weighted to form updated data 214′) at step 310 and coded according to the selected ontology 324 to form updated coded data 335′ at step 330. The initial ontology space 346 is populated with ontological vectors in the updated coded data 335′ at step 340 to augment the initial ontology space 346 and form an updated ontology space 346′. The optimization algorithm identifies updated hypotheses 268′ in the updated ontology space 346′ (and ranks those updated hypotheses 268′) at step 350 as described above. Those updated hypotheses 268′ may be filtered at step 360 as described above.

Referring back to FIG. 2, the initial hypotheses 268 are provided to the dissemination module 280. The hypotheses 268 may include, for example, locally vulnerable and seemingly resistant population segments, local population factors potentially representing hitherto unobserved risk and resilience factors, speed of spread in unknown populations, etc. The hypotheses 268 may identify likely (pharmaceutical and/or nonpharmaceutical) public health interventions relevant for local populations. The hypotheses 268 may identify the likely impacts on local healthcare organizations, such as the need for field hospitals/care centers, the requirement of medical supplies (such as personal protective equipment), supply chain dynamics, etc. Via the dissemination module 280, these respective healthcare organizations may be forewarned of potential impending crisis, and they, in turn, can commence precautionary measures.

To use a specific example, the initial hypotheses 268 identified in the ontology space 346 populated by the ontological vectors identified in the initial data 214 may include:
Viral disease causes pneumonia in persons >70
Bacterial illness causes death in persons >60
Influenza-like illness causes unknown in persons <50

The dissemination module 280 distributes the prediction 246 generated by the predictive model 242 and the hypotheses 268 generated by the hypothesis generation module 260 to the relevant stakeholders and policy makers in the field of infectious disease. The dissemination module 280 may be any software program suitably configured to distribute information (using text, charts, graphics, etc.). The dissemination module 280 may include one or more specialized dashboards (for example, dashboards similar to those described in U.S. patent application Ser. No. 17/059,985, which is incorporated by reference). The distribution module 280 may be, for example, a web server that publishes one or more websites viewable via the client devices 180 over the one or more networks 130 using a web browser. Additionally, or alternatively, the distribution module 280 includes an email server configured to output email messages. The dissemination module 280 may include security features to securely disseminate information (e.g., the hypotheses 268 and the prediction 246) only to authorized users. Additionally, or alternatively, the distribution module 280 may publish information and make that information viewable to the public via the Internet.

### Evaluating the Initial Hypotheses 268 and Identifying Newly Emerging Hypotheses 268

FIG. 4 is a diagram of the system 200 of FIG. 2, at a later point in time, generating and distributing the updated hypotheses 268′ and an updated prediction 246′ according to an exemplary embodiment.

As shown in FIG. 4 and described above with reference to FIG. 3B, the data collection module 210 receives updated data 214′ and the validation/weighting module 220 validates and assigns a weight to each document in the updated data 214′. The predictive model 242 generates an updated prediction 246′ based on the updated data 214′. The updated prediction 246′ is provided to the dissemination module 280 for distribution. The updated data 214′ and updated prediction 246′ are provided to the hypothesis generation module 260. Using the updated data 214′ and updated prediction 246′, the hypothesis generation module 260 populates an updated ontology space 346′ and generates updated hypotheses 268′.

A hypothesis space difference evaluation module 490 compares the updated hypotheses 268′ to the initial hypotheses 268. For example, the hypothesis space difference evaluation module 490 determines whether updated hypotheses 268′ identified in the updated data 214′ were previously identified in the initial data 214 and, if so, may compare the rankings assigned to corresponding initial and updated hypotheses 268 and 268′ by the optimization algorithm and/or the weights of the ontology vectors in the initial and updated ontology spaces 346 and 346′ corresponding to those initial and updated hypotheses 268 and 268′. If an updated hypothesis 268′ was not previously identified in the initial data 214—or if the corresponding initial hypothesis 268 was ranked lower than the updated hypothesis 268′ because the ontology vector in the initial ontology space 346 corresponding to the initial hypothesis 268 was lower weighted than the ontology vector in the updated ontology space 346′ corresponding to the updated hypothesis 268′— then the updated hypothesis 268′ represents a new insight that may help understand, control, and treat the disease.

Similarly, the hypothesis space difference evaluation module 490 determines whether initial hypotheses 268 identified in the initial data 214 are also identified in the updated data 214′ and, if so, may compare the rankings assigned to corresponding initial and updated hypotheses 268 and 268′ and/or the weights of the corresponding ontology vectors. A determination that an initial hypothesis 268 is not identified in the updated data 214′— or corresponds to an updated hypothesis 268′ that is lower ranked and lower weighted than the initial hypothesis 268—is evidence that the initial hypotheses 268 represents an assumption that may no longer be supported by the latest data 214′.

The updated hypotheses 268′, relative to the initial hypotheses 268, can then be delivered to users for consideration and further investigation. Accordingly, the system 200 can be used to inform public health officials and medical practitioners if the newly received data 214′ suggests new inferences about the characteristics of the disease, the effectiveness of medical and/or public health interventions, the impacts on healthcare organizations in geographic areas, etc. Perhaps even more critically, the system 200 can also be used to inform those officials and practitioners if the newly received data 214′ challenges or contradicts previous inferences drawn from earlier data 214. Since the hypotheses 268 and 268′ are combinations of English words, the new hypotheses 268′ identified by the system 200 (and the previous hypotheses 268 challenged or contradicted by the system 200) are immediately understandable to human users. Meanwhile, the hypotheses 268 and 268′ identified by the system 200 can be traced back to the data 214 or 214′ from which those hypotheses 268 and 268′ were identified, enabling public health and medical researchers to evaluate those data sources.

### Using the Difference Between Newly Identified and Previous Hypotheses for Optimization

As described above, if additional ontological vectors (that were not detected in the initial data 214) are identified in the updated data 214′ (e.g., previously unexhibited symptoms, unaffected geographical regions, etc.), the system 200 augments the initial ontology space 346 (that generated the initial hypotheses 268) to form the updated ontology space 346′, which generates the updated hypotheses 268′. In addition to better informing officials, practitioners, and policymakers, the difference between the updated hypotheses 268′ and the initial hypotheses 268 (as determined by the hypothesis space difference evaluation module 490) can also be used by the optimization algorithm described above to more efficiently and effectively identify and rank hypotheses using future data.

For instance, if the set of updated hypotheses 268′ subsumes the set of initial hypotheses 268, then the updated ontology space 346′ subsumes the initial ontology space 346 and only the updated ontology space 346′ needs to be maintained. Accordingly, instances where the set of updated hypotheses 268′ subsume the set of initial hypotheses 268, the system 200 may discard the initial ontology space 346 and augment only the updated ontology space 346′ using future data.

Alternatively, if the set of initial hypotheses 268 subsumes the set of updated hypotheses 268′, then the additional ontological vectors in the updated ontology space 346′ (that were not present in the initial ontology space 346) lead to contradictory or inconsistent hypotheses 268′. In those instances, the additional ontological vectors in the updated ontology space 346′ limit the possibility of identifying valid hypotheses 268′. Also, if those additional ontological vectors are used as the basis for public health regulations, those regulations will be overly restrictive and unsupported by the data 214 and 214′. Accordingly, in instances where the set of initial hypotheses 268 subsumes the set of updated hypotheses 268′, the system 200 may discard additional ontological vectors in the updated ontology space 346′ that were not present in the initial ontology space 346 (or assign those additional ontological vectors lower weights than to the ontological vectors in both the initial ontology space 346 and the updated ontology space 346′).

Finally, if the set of initial hypotheses 268 is equal to the set of updated hypotheses 268′, then the additional ontological vectors in the updated ontology space 346′ (that were not present in the initial ontology space 346) are redundant and may be removed by the system 200.

Returning back to the specifical example above, the difference between the updated hypotheses 268′ and the initial hypotheses 268 may reveal:
A new hypothesis 268′ identified in the updated ontology space 346′ that was not present in the initial ontology space 346, such as:
   Viral disease causes rash in persons >15
A persistent hypothesis 268 identified in the initial ontology space 346 that remains in the updated ontology space 346′, such as:
   Bacterial illness causes death in persons >60 Anomalous hypotheses 268′, such as:
Viral disease causes pneumonia in persons >70
   Viral disease causes pneumonia in persons >20
   Influenza like illness causes unknown in persons <50
   Influenza like illness causes unknown in persons <10

### Using Newly Identified and Recently Evaluated Hypotheses to Update the Predictive Model

As described above, identifying new hypotheses 268′ in newly received data 214′ can help medical practitioners and public health officials identify additional medical and public health interventions that may treat and control the spread of a disease. Also, determining whether initial hypotheses 268 continue to be suggested by the latest data 214′ helps those practitioners and officials evaluate whether the interventions that are currently being implemented are as effective as originally assumed.

Additionally, identifying new hypotheses 268′ (and discarding previous hypotheses 268 that are no longer suggested by the latest data 214′) can help predictive models more accurately predict the future spread of a disease by providing those predictive models with the latest understanding of characteristics of the disease and the effectiveness of various interventions. Accordingly, if the updated hypotheses 268′ significantly differ from the initial hypotheses 268, the system 200 uses those updated hypotheses 268′ to inform the predictive model 242 generated by the machine learning module 240.

As described above, the predictive model 242 predicts the future spread of the disease based on predictor variables identified in the data 214 (e.g., numerical metrics, Boolean conditions, etc.) and associations (e.g., weights, Bayesian probabilities, etc.) between those predictor variables and the future spread of the disease. The machine learning module 240 is trained using the initial data 214 to learn the predictor variables that are associated with the future spread of the disease and the extent of those associations. As updated data 214′ are received, however, new hypotheses 268′ in the updated data 214′ that were not detected in the initial data 214 (e.g., previously unexhibited symptoms, unaffected geographical regions, etc.) may identify additional predictor variables in the updated data 214′ that, if incorporated in the predictive model 242, would improve the accuracy of the predictive model 242. Similarly, new hypotheses 268′ in the updated data 214′ may suggest adjustments to the associations (e.g., weights, Bayesian probabilities, etc.) used by the predictive model 242, which were initially learned by the machine learning module 240 while being trained using the initial data 214, to better reflect the updated hypotheses 268′ in the updated data 214′. By contrast, an initial hypothesis 268 (identified in the initial data 214) failing to appear in the updated data 214′ (or having significantly less weight in the updated ontology space 346′ relative to the initial ontology space 346) is an indication that the initial hypothesis 268 is less relevant than the initial data 214 suggested. Accordingly, the predictive model 242 may be updated to discount that initial hypothesis 268, for example by reducing the weight (or adjusting the probability) previously applied to a variable that the initial hypothesis 268 suggested was predictive of the future spread of the disease (or no longer using that variable at all when generating predictions 246).

In some embodiments, the machine learning module 240 is trained on the newly identified hypotheses 268′ (and/or indications that previously identified hypotheses 268 should be discounted) to learn adjusted associations and/or additional predictor variables indicative of those newly identified hypotheses 268′, as well as variables (previously viewed as predictive) that can be de-weighted or no longer considered. Alternatively, the machine learning module 240 may be trained on the updated hypotheses 268′ to generate a new predictive model 242 to replace the predictive model 242 generated using the initial data 214. In either embodiment, providing the machine learning module 240 with the difference between the updated hypotheses 268′ and the initial hypotheses 268 enables the machine learning module 240 to perform back propagation and readjust the predictor variables and associations (and/or the model structure, initial conditions, boundary conditions, etc.) to make the predictive model 242 represent and classify the current state of knowledge.

To adjust the predictive model 242, for example, the machine learning module 240 may utilize deep learning, for instance with attention on more recent data 214′ and/or on data 214 that are higher weighted by the validation/weighting module 220 to support greater intuition regarding classification results derived by deep learners and/or graph-oriented models to provide interpretability via derivation graphs.¹⁹ ¹⁹ See, e.g., U.S. Pat. No. 11,238,966 to Frieder et al.

In addition to improving the accuracy of the predictive model 242, providing the machine learning module 240 with updated hypotheses 282′ that better reflect the latest understanding of the disease enables the predictive model 242 to generate predictions 246′ that are tailored to local geographic areas based on predictor variables that are specific to those geographic areas (e.g., the current disease metrics in those areas, the demographic composition of those areas, whether public health interventions are required and the level of compliance in those areas, etc.) and the associations between those predictor variables and the spread of the disease suggested by the updated hypotheses 282′.

As the disease continues to spread, the system 200 repeatedly captures updated data 214′ to generate updated hypotheses 268′ and uses those updated hypotheses 268′ to update the predictive model 262. While the process performed by the system is logically viewed as sequential, the data collection, analytics, and dissemination can overlap, either pairwise or in totality. That is, partial analysis and partial dissemination may occur while additional data collection and analysis proceeds.

By combining hypothesis generation/testing and predictive modeling, the disclosed system 200 provides important technical benefits that cannot be realized using separate hypothesis generation systems and predictive models. As described above, prior art predictive models often rely on assumptions to model pandemic infections,²⁰ such as assumptions about the characteristics of a disease, the effectiveness of medical and/or public health interventions, potential changes in human behavior over the prediction period, etc. If the assumptions embedded in those prior art predictive models are inaccurate, those inaccurate assumptions will negatively impact the accuracy of every subsequent prediction generated by those prior art predictive models, even as those prior art predictive models incorporate new data, until those prior art predictive models are updated to no longer rely on those assumptions. Critical in early-stage diagnostic predictions is the ability to forget or “be forgotten.”. Prior art predictive models are either insufficiently powerful to learn the associations needed to derive the ranked hypotheses 268 described above or do not provide sufficient intuition to enable change, including replacing variables and associations previously considered predictive or simply forgetting those previously considered variables. ²⁰ See Cramer et al., supra, wherein seven probabilistic COVID-19 forecasts made explicit assumptions that social distancing and other behavioral patterns would change over the prediction period.

By contrast, the disclosed system 200 uses initial data 214 to generate a predictive model 242 that outputs an initial prediction 246 and then evaluates the assumptions embedded in that predictive model 242 by repeatedly collecting updated data 214′, identifying the hypotheses 268′ in the new data 214′, and comparing those updated hypotheses 268′ to the initial hypotheses 268 identified in the initial data 214 used to generate the predictive model 242. Accordingly, as new data 214′ emerge that challenge or contradict the assumptions embedded in the predictive model 242, the disclosed system 200 is configured to adjust the predictive model 242 to more accurately reflect the most recent understanding of the disease and the public health and medical interventions to control and treat the disease.²¹ ²¹ See Sridhar et al., supra, wherein some early COVID-19 models did not consider the possible effects of mass “test, trace, and isolate” strategies or potential staff shortages on transmission dynamics.

Additionally, while prior art machine learning algorithms can use newly received data to identify unexpected predictor variables and the associations between those predictor variables and potential outcomes, those prior art deep learning algorithms fail to provide any insight as to why predictions change over time. Accordingly, rather than merely identifying numerical metrics based on their fit to past data, the disclosed system 200 goes a step further by coding the new data 214′ (including textual information, etc.) according to an ontology 324, organizing the coded data 335 in an ontology space 346, and using an optimization algorithm to identify and rank hypotheses 268′ found in the new data 214′. In doing so, the system 200 provides human-comprehensible reason(s) for each suggested update to the predictive model 242 and human-comprehensible actions (e.g., public health or clinical interventions) that can be implemented to better control and/or treat the disease (and, therefore, generate predictions 246 that are reflective of more desirable health outcomes). Accordingly, the disclosed system 200 enables researchers to identify the change to our understanding of the disease that triggers each change to the predictive model 242 and, for instance, the probability that each change is permanent, the predicted duration of any change believed to be transient, the likelihood that any change will be repeated, whether any change can be mitigated via a public health or clinical intervention, the probability that a suggested intervention will mitigate the identified issue, other issues that may be caused by the suggested intervention, etc. Those new insights, in addition to their value for keeping public officials and clinicians better informed, also enable the machine learning module 240 to more accurately predict the current trajectory of a disease and the effectiveness of current and potential interventions.

### Example

An illustrative and instructive case in early days of the COVID-19 pandemic concerns the use of masks as a public health intervention to slow/mitigate spread.

Early in the pandemic, masks were not thought to be an effective public health or personal protection measure. On Jan. 29, 2020, the World Health Organization (WHO) noted that “a medical mask is not required, as no evidence is available on its usefulness to protect non-sick persons.”²² That continued to be the definitive guidance on mask wearing. A month later, on Feb. 26, 2020, the U.S. Centers for Disease Control and Prevention (CDC) confirmed the first likely instance of community spread of COVID-19 in the U.S.²³ On Feb. 27, 2020, in a Congressional hearing, CDC Director Robert Redfield was asked whether healthy people should wear a face covering and responded “No.”²⁴ Additional official guidance was disseminated by U.S. Surgeon General Jerome Adams on Feb. 29, 2020. On Twitter, Adams urged Americans to “STOP BUYING MASKS!”, asserting that masks are “NOT effective in preventing general public from catching coronavirus” and that rushing to buy masks would deplete mask supplies for healthcare providers. Indeed, the former assertion had some evidence in the research literature, which presented mixed results in evaluations of the effectiveness of masks in preventing community respiratory illness. ²² https://apps.who.int/iris/handle/10665/330987²³ https://www.cdc.gov/media/releases/2020/s0226-Covid-19-spread html²⁴ https://www.c-span.org/video/?469566-1/house-hearing-coronavirus-response

On Feb. 29, 2020, then-Vice President Pence, speaking as head of the coronavirus task force at a White House press conference, noted that the “average American does not need to go out and buy a mask.” The message was so consistent and ubiquitous that, a week later, on Mar. 8, 2020, Anthony Fauci said in a 60 Minutes interview that “there's no reason to be walking around with a mask,” adding that he was not “against masks” but rather was worried about health care providers and sick people “needing them.” He also mentioned possible “unintended consequences” of mask wearing, including people touching their face frequently when adjusting their masks, posing contamination hazards to themselves.

Given an ontology 324 of respiratory infection and public health interventions, an ontology space 346 populated using data 214 that includes documents corresponding to COVID-19 (such as those alluded to above and others) would have resulted in clusters reflecting that guidance (that masks were not an effective public health or personal protection measure), which was offered universally (outside of China) at this stage of the pandemic. However, applying the hypothesis generation process to the issue of appropriate interventions based on what was known about respiratory infections may have revealed and challenged the (obvious) conflict between the assertion that masks were unlikely to be effective at preventing or slowing community disease with the assertion that they needed to be conserved for healthcare workers, who would be protected by wearing them.

As more was learned, guidance changed. Importantly, in March and April 2020, evidence emerged implicating asymptomatic and presymptomatic transmission of COVID-19 and the implications for mask wearing were recognized. On March 29, former Food and Drug Administration Commissioner Scott Gottlieb published a paper outlining a “roadmap” for emerging from widespread “lockdowns.” Mask use was a prominent recommendation. “Face masks will be most effective at slowing the spread of SARS-CoV-2 if they are widely used, because they may help prevent people who are asymptomatically infected from transmitting the disease unknowingly.”²⁵ ²⁵ https://www.aei.orgkesearch-productskeport/national-coronavirus-response-a-road-map-to-reopening/and

On Mar. 31, 2020, Fauci said he was in “very active discussion” with health officials about reversing guidance on mask use when the U.S. gets in a “situation” where it has a sufficient mask supply, alluding to the emerging evidence that COVID-19 spreads via the air among asymptomatic people who do not cough or sneeze. On Apr. 3, 2020, the CDC updated its guidance on masks and facial coverings, recommending wearing facial coverings “in public settings when around people outside their household, especially when social distancing measures are difficult to maintain.” The WHO followed suit on Apr. 6, 2020, citing presymptomatic transmission and noting that “The use of masks is part of a comprehensive package of prevention control measures that can limit the spread of certain respiratory viral diseases, including COVID-19.”²⁶ ²⁶ World Health Organization, Advice on the use of masks in the context of COVID-19, 1 Dec. 2020, https://www.who.int/publications/i/item/advice-on-the-use-of-masks-in-the-community-during-home-care-and-in-healthcare-settings-in-the-context-of-the-novel-coronavirus-(2019-ncov)-outbreak

Applying hypothesis generation methods to the biomedical and epidemiology literature during this period or before would have revealed the appearance of new clusters in ontology space 346 corresponding to these new data (regarding transmission mechanisms). Specifically, the emergence of evidence implicating viral shedding in respiratory droplets before COVID-19 symptoms appeared immediately suggests the importance of intervention measures such as mask wearing among others for the general population. The appearance of such hypotheses 268 in the ontology space 346 could have cued a search for implications much more quickly than happened, perhaps even instantaneously if the ontology 324 was sufficiently connected or linked to control measures.

Guidance remained consistent for a time of low transmission but then became more important as a new wave hit. The U.S. saw a dramatic acceleration of COVID-19 transmission in the fall of 2020. In the pre-COVID-vaccine era, nonpharmaceutical interventions (NPIs) continued to be the only means available to prevent increasing morbidity and mortality. Modeling and other epidemiology studies became available implicating the importance of such NPIs for the coming wave. On Oct. 14, 2020, Fauci, discussing the upcoming holidays and associated dangers of the cold weather, said “Don't be afraid to wear a mask in your house if you're not certain that the persons in the house are negative.”²⁷ He reiterated that advice more strongly roughly a week later, saying “ . . . if people are not wearing masks, then maybe we should be mandating it”²⁸ in a CNN interview. “There's going to be a difficulty enforcing it, but if everyone agrees that this is something that's important and they mandate it, and everybody pulls together and says, you know, ‘we're going to mandate it but let's just do it,’ I think that would be a great idea to have everybody do it uniformly.”²⁷ CBS News, Dr. Fauci on COVID surge, Trump's recovery, holiday travel and more—Full interview, 14 Oct. 2020, https://www.cbsnews.com/video/dr-fauci-on-covid-surge-trumps-recovery-holiday-travel-and-more-full-interview/²⁸ CNN, Fauci says it might be time to mandate masks as Covid-19 surges across US, 23 Oct. 2020, https://www.cnn.com/2020/10/23/health/fauci-covid-mask-mandate-bn/index.html

Applying the ontology 324 described above to the continuing epidemiology and biomedical literature among other sources during this period would have detected new clusters in the ontology space 324 surrounding efficiency and performance of masks and mask type, calling attention to the importance of mask material and mask-use strategies. Meanwhile, a predictive model 242 adjusted to reflect the newly recognized correlation between mask usage/materials and lower transmission rates would have estimated the public health benefit of those interventions and illustrated their importance.

As the most intense wave of the pandemic in the U.S. receded, due in no small part to the appearance of an effective vaccine, new guidance was published on double masking. On Feb. 10, 2021, the CDC released research finding that wearing a cloth mask over a surgical mask offers more protection against the coronavirus, as does tying knots on the ear loops of surgical masks. The lateness of this and other updated guidelines is tragic and could have been issued earlier if learning had occurred more rapidly, as outlined in the method of this application.

During the summer and early fall of 2021, numerous documents in the literature have examined the importance of not only vaccination, and not only masking in prevention of COVID-19, which began increasing over the summertime, but combining those measures. Using the hypothesis generation module 260, that data 214 would undoubtedly result in additional clusters in ontology space 324 and should cue policy guidance to the public that the need for wearing a mask has not yet passed.

While preferred embodiments have been set forth above, those skilled in the art who have reviewed the present disclosure will readily appreciate that other embodiments can be realized within the scope of the invention. For example, disclosures of specific numbers of hardware components, software modules and the like are illustrative rather than limiting. Accordingly, the present invention should be construed as limited only by any appended claims.

What is claimed is:

1. A method for identifying hypotheses regarding a pandemic infection in initial data and testing the identified hypotheses using updated data, the method comprising:
receiving initial data from a plurality of data sources;
using the initial data to generate and rank initial hypotheses regarding a pandemic infection by:
coding the initial data according to an ontology having ontological vectors, each ontological vector corresponding to a hypothesis, by identifying each of the ontological vectors in the initial data; and
using an optimization algorithm to rank the ontological vectors identified in the initial data;
receiving updated data;
using the updated data to generate and rank updated hypotheses by identifying the ontological vectors in the updated data and ranking the ontological vectors identified in the updated data; and
comparing the updated hypotheses to the initial hypotheses by:
identifying an updated hypothesis having a higher ranking than an initial hypothesis corresponding to the same ontological vector; or
identifying an initial hypothesis having a higher ranking than an updated hypothesis corresponding to the same ontological vector.

2. The method of claim 1, wherein:
the ontology comprises a plurality of elements and each element comprises a plurality of ontological terms;
each ontological vector comprises an ontological term from each of two or more of the plurality of elements; and
coding the initial data according to the ontology comprises:
forming an initial ontology space wherein each dimension of the ontology space comprises one or more of the elements of the ontology; and
populating the ontology space by adding the ontological vectors identified in the initial data such that a weight or each point in the ontology space is proportional to a number of ontological vectors associated with that point found in the initial data.

3. The method of claim 2, wherein using the optimization algorithm to rank the ontological vectors identified in the initial data comprises:
using the optimization algorithm to rank points or clusters of points in the ontology space based on the weights of the points or the clusters of points; and
outputting a ranked list of initial hypotheses, each initial hypothesis corresponding to one of the points or clusters of points in the ontology space.

4. The method of claim 1, wherein using the optimization algorithm to rank the ontological vectors comprises:
using a first optimization function to perform a coarse ranking of the ontological vectors and identify a subset of the highest ranked ontological vectors; and
using a second optimization function to perform a precise ranking of the subset of ontological vectors ranked highest by the first optimization function.

5. The method of claim 1, wherein the optimizing algorithm includes a heuristic optimization function or an iterative optimization function.

6. The method of claim 1, further comprising:
using the initial data to train a machine learning module to generate a predictive model of a pandemic infection, the predictive model generating an initial prediction of how a disease will spread in one or more locations;
updating the predictive model based on the comparison of the updated hypotheses and the initial hypotheses; and
using the updated data and the updated predictive model to generate an updated prediction of how the disease will spread.

7. The method of claim 6, wherein the predictive model generates the initial prediction based on predictor variables, identified in the initial data by the machine learning module, and associations, identified by the machine learning module, between the identified predictor variables and the spread of the disease.

8. The method of claim 7, wherein the machine learning module adjusts the predictive model by learning additional predictor variables and/or adjusted associations between the identified predictor variables and the spread of the disease.

9. The method of claim 7, wherein the associations used by the predictive model comprise weights or Bayesian probabilities.

10. The method of claim 7, wherein the predictor variables used by the predictive model comprise numerical values or Boolean conditions.

11. The method of claim 1, further comprising:
outputting, for transmittal via one or more computer networks:
the updated hypothesis having a higher ranking than the initial hypothesis corresponding to the same ontological vector; or
the initial hypothesis having a higher ranking than the updated hypothesis corresponding to the same ontological vector.

12. The method of claim 11, wherein:
the updated hypothesis having a higher ranking than the initial hypothesis corresponding to the same ontological vector represents a potential new insight regarding the pandemic infection; or
the initial hypothesis having a higher ranking than the updated hypothesis corresponding to the same ontological vector represents a previous assumption regarding the pandemic infection.

13. A system for identifying hypotheses regarding a pandemic infection in initial data and testing the identified hypotheses using updated data, the system comprising:
a data collection module that receives initial data from a plurality of data sources and later receives updated data;
a hypothesis generation module that:
generates initial hypotheses by coding the initial data according to an ontology having ontological vectors, identifies the ontological vectors in the initial data, and uses an optimization algorithm to rank the ontological vectors identified in the initial data; and
generates updated hypotheses by identifying and ranking the ontological vectors in the updated data; and
a hypothesis space difference evaluation module that comparing the updated hypotheses to the initial hypotheses and:
identifies an updated hypothesis having a higher ranking than an initial hypothesis corresponding to the same ontological vector; or
identifies an initial hypothesis having a higher ranking than an updated hypothesis corresponding to the same ontological vector.

14. The system of claim 13, wherein:
the ontology comprises a plurality of elements and each element comprises a plurality of ontological terms;
each ontological vector comprises an ontological term from each of two or more of the plurality of elements; and
the hypothesis generation module codes the initial data according to the ontology by:
forming an initial ontology space wherein each dimension of the ontology space comprises one or more of the elements of the ontology; and
populating the ontology space by adding the ontological vectors identified in the initial data such that a weight or each point in the ontology space is proportional to a number of ontological vectors associated with that point found in the initial data.

15. The system of claim 14, wherein the hypothesis generation module uses the optimization algorithm to rank the ontological vectors by:
using the optimization algorithm to rank points or clusters of points in the ontology space based on the weights of the points or the clusters of points; and
outputting a ranked list of initial hypotheses, each initial hypothesis corresponding to one of the points or clusters of points in the ontology space.

16. The system of claim 13, wherein the hypothesis generation module uses the optimization algorithm to rank the ontological vectors by:
using a first optimization function to perform a coarse ranking of the ontological vectors and identify a subset of the highest ranked ontological vectors; and
using a second optimization function to perform a precise ranking of the subset of ontological vectors ranked highest by the first optimization function.

17. The system of claim 13, wherein the optimizing algorithm includes a heuristic optimization function or an iterative optimization function.

18. The system of claim 13, further comprising:
a machine learning module trained on the initial data to generate a predictive model of a pandemic infection, the predictive model generating an initial prediction of how a disease will spread in one or more locations,
wherein the machine learning module updates the predictive model based on the comparison of the updated hypotheses and the initial hypotheses; and
the updated predictive model uses the updated data and to generate an updated prediction of how the disease will spread.

19. The system of claim 18, wherein the predictive model generates the initial prediction based on predictor variables, identified in the initial data by the machine learning module, and associations, identified by the machine learning module, between the identified predictor variables and the spread of the disease.

20. The system of claim 19, wherein the machine learning module adjusts the predictive model by learning additional predictor variables and/or adjusted associations between the identified predictor variables and the spread of the disease.
